(19) United States
(12) Patent Application Publication
(10) Pub. No.: US 2025/0255990 A1
(43) Pub. Date: Aug. 14, 2025

(54) FLUORESCENT NANODIAMOND COATED WITH POROUS POLYDOPAMINE, AND USE THEREOF

(71) Applicant: KOREA RESEARCH INSTITUTE OF STANDARDS AND SCIENCE, Daejeon (KR)

(72) Inventors: Haksung JUNG, Daejeon (KR); Jeong Hyun SHIM, Daejeon (KR); Mina LEE, Daejeon (KR); Sihwa JOO, Daejeon (KR)

(73) Assignee: KOREA RESEARCH INSTITUTE OF STANDARDS AND SCIENCE, Daejeon (KR)

(21) Appl. No.: 19/095,112

(22) Filed: Mar. 31, 2025

Related U.S. Application Data
(63) Continuation of application No. PCT/KR2023/000689, filed on Jan. 13, 2023.

(30) Foreign Application Priority Data
Sep. 30, 2022 (KR) 10-2022-0125088

(51) Int. Cl. A61K 49/00; A61K 41/00; B82Y 5/00; B82Y 20/00; B82Y 40/00; C09K 11/02; C09K 11/65

(52) U.S. Cl. CPC A61K 49/0019; A61K 41/0052; C09K 11/025; C09K 11/65; B82Y 5/00; B82Y 20/00; B82Y 40/00

(57) ABSTRACT

The present invention relates to fluorescent nanodiamond coated with porous polydopamine, a method for preparing the fluorescent nanodiamond, and novel use of the fluorescent nanodiamond. The fluorescent nanodiamond coated with porous polydopamine, of the present invention, has excellent colloidal stability and biocompatibility through a second-order reaction, and thus can be used in various biological application fields such as those of lesion labelling, drug delivery or miRNA detection.

### CROSS-REFERENCE TO RELATED PATENT APPLICATION

This application is a Continuation of International Application No. PCT/KR2023/000689, filed on Jan. 13, 2023, which claims the benefit of Korean Patent Application No. 10-2022-0125088, filed on Sep. 30, 2022, in the Korean Intellectual Property Office, the disclosure of which is incorporated herein in its entirety by reference.

### TECHNICAL FIELD

The present disclosure relates to a fluorescent nanodiamond coated with porous polydopamine, a method for preparing the fluorescent nanodiamond, and a novel use of the fluorescent nanodiamond.

### BACKGROUND ART

Fluorescent nanodiamond (FND) is a material that has attracted attention in biomedical applications due to its unique and excellent physicochemical properties. Optically pumped FND causes a transition of electron spin states from a triplet ground state to a triplet excited state (mₛ=0 and mₛ=±1). During the transition from the excited state back to the ground state, photons in a near-infrared region (670 to 890 nm, approximately 70%) are emitted. Due to its emission in the near-infrared region with deep penetration depth, it is known to be suitable for in vivo imaging applications.

Since the fluorescence lifetime of FND is relatively long compared to the autofluorescence of cells and tissues, it can be used for fluorescence lifetime imaging. Unlike conventional organic dyes, the fluorescence of FND does not exhibit irregular fluctuations in brightness over time. In addition, FND has no photobleaching effect, so initial fluorescence intensity is stably maintained. These optical properties of FND are derived from nitrogen-vacancy (NV) centers existing in an FND lattice structure. Nitrogen substituted for carbon atoms is the most common impurity in diamond. When nitrogen impurity-containing diamond is irradiated with a high-energy electron beam and then heat-treated at a high temperature, nitrogen-vacancy centers are created.

FND has excellent optical and physicochemical properties, but its tendency to aggregate in saline solutions limits its biological application, and its inert surface properties make effective surface functionalization challenging as well. In order to be widely used in biomedical applications such as imaging, drug delivery, sensing, and therapy, the aforementioned problems need to be solved.

Under this background, the inventors of the present disclosure completed the present disclosure by confirming that the fluorescent nanodiamond coated with porous polydopamine, prepared by coating the FND surface with porous polydopamine, may maintains high colloidal stability even in saline solutions through various second-order reactions, thus enabling its application in various biomedical applications.

### DISCLOSURE

### Technical Problem

The present disclosure aims to solve the above-mentioned problems and other problems related thereto.

An aspect of the present disclosure is to provide a fluorescent nanodiamond coated with porous polydopamine.

Another aspect of the present disclosure is to provide a method for preparing the fluorescent nanodiamond.

Another aspect of the present disclosure is to provide a composition for thermotherapy comprising the fluorescent nanodiamond as an active ingredient.

Another aspect of the present disclosure is to provide a composition for drug delivery comprising the fluorescent nanodiamond.

Another aspect of the present disclosure is to provide a composition for lesion labeling comprising the fluorescent nanodiamond.

Another aspect of the present disclosure is to provide a composition for detecting nucleic acids comprising the fluorescent nanodiamond.

Another aspect of the present disclosure is to provide a method for thermotherapy, comprising administering to a subject a composition comprising the fluorescent nanodiamond as an active ingredient.

Another aspect of the present disclosure is to provide a use of the fluorescent nanodiamond for thermotherapy.

Another aspect of the present disclosure is to provide a use of the fluorescent nanodiamond for drug delivery.

Another aspect of the present disclosure is to provide a use of the fluorescent nanodiamond for lesion labeling.

Another aspect of the present disclosure is to provide a use of the fluorescent nanodiamond for nucleic acid detection.

Another aspect of the present disclosure is to provide a composition for preventing, treating, or diagnosing a disease, comprising the fluorescent nanodiamond as an active ingredient.

Another aspect of the present disclosure is to provide a use of the fluorescent nanodiamond for the prevention, treatment, or diagnosis of a disease.

The technical problems to be achieved in accordance with the technical ideas of the present disclosure disclosed in the present specification are not limited to addressing those mentioned above, and other problems not mentioned will be clearly understood by those skilled in the art from the following description.

### Technical Solution

This will be specifically described as follows. Meanwhile, each of the descriptions and embodiments disclosed in the present application may also be applied to other descriptions and embodiments. That is, all combinations of various elements disclosed in the present application fall within the scope of the present application. In addition, the scope of the present application cannot be considered limited by the specific description set forth below.

An aspect of the present disclosure provides a fluorescent nanodiamond coated with porous polydopamine.

As used herein, the term “nanodiamond” refers to a diamond particle of nanometer scale. The nanodiamond includes natural and synthetic diamond obtained from various synthetic processes as well as diamond-like carbon (DLC) in a form of microparticles. The nanodiamond particle may be less than 1 μm, may be in the range of 1 to 999 nm, and may be specifically in the range of 1 to 800 nm, 1 to 500 nm, or 1 to 100 nm, but is not limited thereto. The nanodiamond is commercially available and may be prepared using methods known in the art. The nanodiamond may be prepared by, for example, detonation of a specific explosive in a sealed container, laser ablation, high-energy ball milling of diamond microcrystals, plasma-assisted chemical vapor deposition, or autoclave synthesis from a supercritical fluid.

As used herein, the term “fluorescent nanodiamond (FND)” collectively refers to nanodiamonds that fluoresce when exposed to an absorption spectrum, and may be commercially available or prepared using methods known in the art.

Fluorescent nanodiamonds are a material that has attracted attention in biomedical applications due to their unique and excellent physicochemical properties, but they have difficulty in various biological applications due to their tendency to aggregate in saline solutions. Therefore, the inventors of the present disclosure have overcome the above-mentioned drawbacks by coating mesoporous polydopamine on fluorescent nanodiamond to provide a large surface area and pore size, thereby enabling various biomedical applications such as lesion labeling, miRNA detection, and drug delivery.

The fluorescent nanodiamond coated with porous polydopamine of the present disclosure may exhibit an increased drug delivery efficiency compared to fluorescent nanodiamonds that are not coated with porous polydopamine or fluorescent nanodiamonds coated with non-porous polydopamine.

Specifically, the fluorescent nanodiamond coated with porous polydopamine of the present disclosure has an increased pore size compared to the fluorescent nanodiamond coated with polydopamine, enables enhanced bioimaging or biometric information sensing due to higher fluorescence intensity, and exhibits significantly superior drug delivery efficiency.

In the present disclosure, the porous polydopamine may be macroporous polydopamine, microporous polydopamine, or mesoporous polydopamine, and specifically, may be mesoporous polydopamine (mPDA).

The pore size of the drug delivery vehicle should vary depending on the size of the drug (including protein) that can be loaded, and if an appropriate pore size is not used, it cannot be used as a drug delivery vehicle. In addition, if the pore size is small, the surface area increases, and if the pore size is large, the surface area decreases, so the amount of drug to be loaded may vary depending on the size of the surface area.

In the present disclosure, the fluorescent nanodiamond may be surface-modified with poly(ethylene glycol) methyl ether thiol (mPEG-SH), hyperbranched polyglycerol (HPG), or d-α-tocopheryl polyethylene glycol 1000 succinate (TPGS), and any substance containing an amine or thiol may be used for surface modification without limitation.

Specifically, in an embodiment of the present disclosure, when mesoporous polydopamine-coated fluorescent nanodiamond (FND@mPDA) was surface-modified with mPEG-SH, hyperbranched polyglycerol, or d-α-tocopheryl polyethylene glycol 1000 succinate (TPGS), it was confirmed that when surface-modified with mPEG-SH (FND@mPDA-PEG), the colloidal stability increased and lesion labeling was possible compared to fluorescent nanodiamond (FND) that are not coated with mesoporous polydopamine, when surface-modified with hyperbranched polyglycerol (FND@mPDA-HPG), miRNA detection was possible, and when surface-modified with TPGS (FND@mPDA-TPGS), the drug delivery efficiency increased, thereby achieving more excellent anticancer effect.

In the present disclosure, the “colloidal stability” refers to a property of a specific substance maintaining a state of being dispersed in another substance, and excellent colloidal stability indicates that a specific substance does not aggregate in another substance (e.g., gas, liquid, or solid).

In the present disclosure, the thickness of the porous polydopamine may be 1 nm to 30 nm, 10 nm to 20 nm, and specifically 10 nm to 15 nm, but is not limited thereto.

In an embodiment of the present disclosure, it was confirmed that when the thickness of the mesoporous polydopamine of the FND@mPDA is 13.6 nm, the heat generation induced by laser irradiation increased compared to FND@mPDA having a thickness of 3.9 nm, resulting in a significantly excellent effect on the death of cancer cells.

Another aspect of the present disclosure provides a method for preparing a fluorescent nanodiamond coated with porous polydopamine, comprising:
(a) preparing a mixture by mixing triblock copolymer pluronic F127 (F127) and 1,3,5-trimethyl benzene (TMB);
(b) adding dopamine, fluorescent nanodiamond, and TRIS to the mixture;
(c) ultrasonicating a precipitate obtained by centrifuging the mixture after the addition in step (b); and
(d) redispersing the precipitate in a mixed solution of ethanol and acetone after the ultrasonication in step (c) to obtain a fluorescent nanodiamond coated with porous polydopamine.

In the present disclosure, the concentration of the dopamine may be 1 mM to 3 mM, and specifically 1.5 mM to 3 mM, but is not limited thereto.

In an embodiment of the present disclosure, the shell thickness of the mesoporous polydopamine in FND@mPDA was 13.6 nm when prepared at a dopamine concentration of 1.69 mM, whereas the shell thickness of the mesoporous polydopamine in FND@mPDA was 25.5 nm when prepared at a dopamine concentration of 2.53 mM, confirming that the shell thickness of the mesoporous polydopamine increases with higher added dopamine concentrations.

In the present disclosure, the content of the 1,3,5-trimethyl benzene may be 0.1 mL to 1 mL, specifically 0.3 mL to 0.8 mL, and more specifically 0.4 mL to 0.7 mL.

In the present disclosure, the F127 and TMB of step (a) may be dissolved and mixed in a solution containing deionized water and ethanol.

In the present disclosure, the solution containing deionized water and ethanol may be a mixture of deionized water and ethanol in a volume ratio of 1:1 to 2:1, specifically 1:1 to 1.5:1, and more specifically 1:1 to 1.1:1.

In the present disclosure, the fluorescent nanodiamond of step (b) may be 10 mg to 40 mg, and specifically 10 to 20 mg, but is not limited thereto.

In the present disclosure, the mixed solution of ethanol and acetone of step (c) may be a mixture of ethanol and acetone in a volume ratio of 1:1 to 10:1, specifically 1:1 to 5:1, and more specifically 2:1.

In the present disclosure, the method may further comprise stirring of the mixture before centrifugation in step (c) for 1 to 24 hours to perform the reaction. The shell thickness of the porous polydopamine may be adjusted depending on the reaction time.

Another aspect of the present disclosure provides a composition for thermotherapy comprising the fluorescent nanodiamond coated with porous polydopamine as an active ingredient.

In the present disclosure, the fluorescent nanodiamond coated with porous polydopamine may be surface-modified with mPEG-SH, hyperbranched polyglycerol, or d-α-tocopheryl polyethylene glycol 1000 succinate.

As used herein, the term “thermotherapy” refers to a treatment that involves exposing body tissues to a temperature slightly higher than normal body temperature to kill lesion cells, including cancer cells, or to enhance their sensitivity to radiotherapy or anticancer drugs. According to the present disclosure, the composition of the present disclosure provides a thermotherapy effect by the fluorescent nanodiamond coated with porous polydopamine, thereby achieving a therapeutic effect by photo-induced heating specifically to a desired area without affecting normal tissues and organs in the surrounding area.

The composition for thermotherapy may achieve therapeutic efficacy by direct injection into the body. That is, the composition for thermotherapy comprising the fluorescent nanodiamond coated with porous polydopamine as an active ingredient may generate heat by irradiating light on the fluorescent nanodiamond injected into a target organ, and may kill lesion cells including cancer cells by this heat generation. This treatment method may be performed as a single treatment method or in combination with a conventional treatment method, or as an auxiliary treatment method.

The composition for thermotherapy of the present disclosure is typically provided as a pharmaceutical composition. Therefore, the composition for thermotherapy of the present disclosure comprises a pharmaceutically acceptable carrier. Pharmaceutically acceptable carriers are those conventionally used in formulations, including, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, or mineral oil. Suitable pharmaceutically acceptable carriers and formulations are described in detail in Remington's Pharmaceutical Sciences, 19th ed., 1995.

The composition for thermotherapy of the present disclosure is preferably administered parenterally. In a case of parenteral administration, it can be administered by intravenous injection, subcutaneous injection, intramuscular injection, intraperitoneal injection, or intralesional injection. The appropriate dosage of the composition of the present disclosure may be prescribed in various ways depending on factors such as the formulation method, administration method, patient's age, weight, sex, pathological condition, food, administration time, administration route, excretion rate, and response sensitivity. The composition for thermotherapy of the present disclosure comprises a fluorescent nanodiamond coated with porous polydopamine, which exhibits a therapeutically effective amount of photo-induced thermogenic effect. The term “therapeutically effective amount” refers to an amount sufficient to treat the targeted disease for therapeutic purpose, and is generally 0.0001 to 100 mg/kg.

The composition for thermotherapy of the present disclosure is typically provided as a pharmaceutical composition. The pharmaceutical composition may be prepared in a unit dosage form by formulating the composition using a pharmaceutically acceptable carrier and/or excipient according to a method that may be easily performed by a person having ordinary knowledge in the art to which the present disclosure belongs, or may be prepared by introducing it into a multi-dose container. Here, the formulation may be in the form of a solution, suspension, or emulsion in an oil or aqueous medium, or in the form of an extract, powder, granule, tablet, or capsule, and may further comprise a dispersant or a stabilizer.

According to a specific embodiment of the present disclosure, the disease treated with the composition for thermotherapy of the present disclosure is cancer (or tumor). The composition for thermotherapy of the present disclosure is particularly useful for treating cancer, and may effectively induce death of cancer cells in various cancer diseases, such as glioblastoma, malignant glioma, breast cancer, lung cancer, colon cancer, anal cancer, astrocytoma, leukemia, lymphoma, head and neck cancer, liver cancer, testicular cancer, cervical cancer, sarcoma, hemangioma, esophageal cancer, eye cancer, laryngeal cancer, oral cancer, mesothelioma, myeloma, oral cancer, rectal cancer, pharyngeal cancer, bladder cancer, uterine cancer, ovarian cancer, prostate cancer, large bowel cancer, pancreatic cancer, kidney cancer, stomach cancer, skin cancer, basal cell carcinoma, melanoma, squamous cell carcinoma, oral squamous cell carcinoma, colorectal cancer, or endometrial cancer.

Another aspect of the present disclosure provides a composition for drug delivery comprising the fluorescent nanodiamond coated with porous polydopamine.

The fluorescent nanodiamond coated with porous polydopamine is as described above.

As used herein, the term “composition for drug delivery” means any form of composition that does not have pharmacological activity by itself or, even if it has pharmacological activity, loads a separate pharmacological component to move to a lesion site or a target cell, thereby further enhancing the pharmacological activity of the loaded pharmacological component.

In the present disclosure, the drug is a broad concept that includes not only conventional drugs but also biological delivery substances. The drug may be any known drug, and for example, the drug may include, but is not limited to, chemical drugs, protein drugs, peptide drugs, nucleic acid molecules for gene therapy, and nanoparticles. Specifically, for example, the drugs includes, but are not limited to, anticancer agents, antidiabetic agents, anti-inflammatory agents, analgesics, antiarthritic agents, antispasmodics, antidepressants, antipsychotics, tranquilizers, antianxiety agents, narcotic antagonists, antiparkinson agents, cholinergic agonists, antiangiogenesis inhibitors, immunosuppressants, antiviral agents, antibiotics, appetite suppressants, analgesics, anticholinergics, antihistamines, antimigraine agents, hormonal agents, coronary vascular, cerebrovascular or peripheral vasodilators, contraceptives, antithrombotic agents, diuretics, antihypertensives, cardiovascular disease treatment agent, cosmetic ingredients (e.g., anti-wrinkle agents, anti-aging agents, and skin whitening agents), etc.

More specifically, the drug comprises an anticancer agent comprising at least one selected from doxorubicin, cisplatin, paclitaxel, vincristine, topotecan, docetaxel, 5-fluorouracil (5-FU), gleevec, carboplatin, daunorubicin, valrubicin, flutamide, and gemcitabine; or an antidiabetic agent comprising insulin or an insulinotropic peptide, but is not limited thereto.

In an embodiment of the present disclosure, a doxorubicin-loaded FND@PDA (FND@PDA-DOX-TPGS) or FND@mPDA (FND@mPDA-DOX-TPGS) surface-modified with TPGS was prepared to compare drug delivery efficiency. As a result, it was confirmed that FND@mPDA-DOX-TPGS had increased drug absorption efficiency and decreased multi-drug resistance (MDR) compared to FND@PDA-DOX-TPGS.

Another aspect of the present disclosure provides a composition for lesion labeling comprising the fluorescent nanodiamond coated with porous polydopamine.

The fluorescent nanodiamond coated with porous polydopamine is as described above.

In the present disclosure, a composition comprising the fluorescent nanodiamond coated with porous polydopamine may be used as a fluorescent probe to mark a lesion. Specifically, by binding the composition to biological tissues such as cancer cells and marking the bonded location, the marked location may be confirmed with the naked eye or using a detection tool.

In an embodiment of the present disclosure, a fluorescent nanodiamond coated with mesoporous polydopamine was surface-modified with mPEG-SH and introduced into HeLa cells. As a result, it was confirmed that the fluorescent nanodiamond was evenly attached to the cells, enabling the detection of a fluorescent signal.

Another aspect of the present disclosure provides a composition for detecting nucleic acids, comprising the fluorescent nanodiamond coated with porous polydopamine.

The fluorescent nanodiamond coated with porous polydopamine is as described above.

In the present disclosure, the nucleic acid may be DNA, RNA, mRNA, or miRNA, but is not limited thereto.

miRNA is a small non-coding single-stranded RNA of 22 nucleotides or less in length and regulates gene expression at the post-transcriptional level. miRNA plays an important role in biological processes such as development, hematopoietic differentiation, regulation, metabolism, apoptosis, and proliferation, and is known as a cancer diagnostic biomarker due to its high correlation with various tumor formations. Therefore, detection of miRNA in vivo is very important for disease diagnosis, treatment, and new drug development.

Conventional miRNA detection using polydopamine is an indirect detection method in which a fluorophore-hybridized oligonucleotide capable of reacting with miRNA is fixed on the surface of polydopamine to suppress fluorescence emission by polydopamine, and when miRNA is present, complementary action causes the hybrid material attached to polydopamine to detach, thereby generating a fluorescence signal. However, this method has drawbacks such as a long detection time and a complicated experimental process. On the other hand, the fluorescent nanodiamond coated with porous polydopamine of the present disclosure may directly detect miRNA, enabling detection of miRNA more easily and quickly.

In an embodiment of the present disclosure, a fluorescent nanodiamond coated with mesoporous polydopamine was surface-modified with hyperbranched polyglycerol and miRNA was introduced at the terminal of the hyperbranched polyglycerol. Subsequently, a molecule capable of specifically binding to a functional group present at the terminal of the miRNA was introduced onto a quartz surface to detect miRNA. As a result, fluorescent spots induced by FND can be observed by selectively binding to each other only in the presence of miRNA, and the number of fluorescence spots of the detected molecules increases as the concentration of miRNA increases. Thus, it was confirmed that FND@mPDA surface-modified with hyperbranched polyglycerol may be used as a fluorescent probe in methods for directly detecting miRNA.

Another aspect of the present disclosure provides a method for thermotherapy, comprising administering to a subject a composition comprising the fluorescent nanodiamond coated with porous polydopamine as an active ingredient.

In the present disclosure, the “thermotherapy” is as described above.

In the present disclosure, the composition used in the method for thermotherapy is a composition for thermotherapy, as described above.

In the present disclosure, the “subject” may be a mammal including a human, a dog, a cat, a cow, a horse, a pig, a rat, etc., but is not particularly limited thereto.

Another aspect of the present disclosure provides a use of the fluorescent nanodiamond coated with porous polydopamine for thermotherapy.

Another aspect of the present disclosure provides a use of the fluorescent nanodiamond coated with porous polydopamine for drug delivery.

Another aspect of the present disclosure provides a use of the fluorescent nanodiamond coated with porous polydopamine for lesion labeling.

Another aspect of the present disclosure provides a use of the fluorescent nanodiamond coated with porous polydopamine for nucleic acid detection.

Another aspect of the present disclosure provides a composition for preventing, treating, or diagnosing a disease, comprising the fluorescent nanodiamond as an active ingredient.

Another exemplary object of the present disclosure provides a use of the fluorescent nanodiamond for the prevention, treatment, or diagnosis of a disease.

### Advantageous Effects

The fluorescent nanodiamond coated with porous polydopamine of the present disclosure, has excellent colloidal stability and biocompatibility, and thus can be applied to various biological applications such as lesion labeling, drug delivery, or miRNA detection.

### BRIEF DESCRIPTION OF DRAWINGS

A brief description of each drawing is provided to more fully understand the drawings cited in this specification.

FIG. 1 shows a synthesis process of a fluorescent nanodiamond coated with porous polydopamine of the present disclosure.

FIG. 2 shows the results confirming the mPDA shell thickness of FND@mPDA according to the concentration of fluorescent nanodiamond.

FIG. 3 shows the results confirming the mPDA shell thickness of FND@mPDA according to the concentration of dopamine.

FIG. 4 shows the results confirming the pore size of FND@mPDA according to the concentration of TMB.

FIG. 5 shows the results confirming the size and zeta potential of FND@mPDA-HPG, FND@mPDA-HPG-Oligo, FND@mPDA-DOX, and FND@mPDA-DOX-TPGS prepared in the present disclosure.

FIG. 6 shows the results of analyzing the weight loss of FND, FND@PDA, and FND@mPDA under a nitrogen environment.

FIG. 7 shows nitrogen adsorption-desorption isotherms of FND@PDA and FND@mPDA.

FIG. 8 shows pore size distributions of FND@PDA and FND@mPDA.

FIG. 9 shows the results of observing the color of a mixture with the naked eye as the reaction time elapsed during the preparing process of FND@mPDA.

FIG. 10 shows the results of measuring UV absorption spectra as the reaction time elapsed during the preparing process of FND@mPDA.

FIG. 11 shows the results of observing mPDA formed on the FND surface as the reaction time elapsed using a transmission electron microscope.

FIG. 12 shows the results of observing the external appearance of FND and FND@mPDA using a transmission electron microscope.

FIG. 13 shows an optical absorption graph of FND and FND@mPDA using UV-vis absorption analysis.

FIG. 14 shows the results of measuring the heat generation according to the concentration by irradiating light on FND@mPDA.

FIG. 15 shows the results of measuring the heat generation according to the thickness of mPDA by irradiating light on FND@mPDA.

FIG. 16 shows the results confirming a doxorubicin loading amount of FND@mPDA over time.

FIG. 17 shows the results comparing the doxorubicin loading amount between FND@PDA and FND@mPDA.

FIG. 18 shows the results comparing the particle sizes of FND, FND@mPDA, and FND@mPDA-PEG.

FIG. 19 shows the results confirming the precipitation of FND and FND@mPDA in PBS buffer.

FIG. 20 shows the results confirming the hydrodynamic radius of FND@mPDA-PEG over time in various aqueous solution environments.

FIG. 21 shows the results of comparing cell viability by treating HeLa cells with FND and FND@mPDA-PEG, respectively (A: cell viability according to treatment concentration, B: cell viability according to treatment time).

FIG. 22 shows the results confirming the effects on cells by treating HeLa cells with doxorubicin, FND, and FND@mPDA-PEG, respectively.

FIG. 23 shows the results confirming the potential for cancer cell labeling based on aggregation by treating HeLa cells with FND and FND@mPDA-PEG, respectively.

FIG. 24 shows a hybridization process through binding reaction of FND@mPDA-HPG-Oligo and miRNA.

FIG. 25 shows the SMC TIRF microscopy-based setup for quantifying miRNA.

FIG. 26 shows the results of fluorescence spots detected by FND@mPDA surface-modified with hyperbranched polyglycerol according to miRNA concentration.

FIG. 27 shows the results of comparing the uptake of FND@PDA-DOX-TPGS or FND@mPDA-DOX-TPGS in MDA-MB-231 cells using a reflection dark-field microscope.

FIG. 28 shows the results of comparing the uptake of FND@PDA-DOX-TPGS or FND@mPDA-DOX-TPGS in MDA-MB-231 cells using a bright-field image.

FIG. 29 shows the results confirming cytotoxicity by treating MDA-MD-231 cells with DOX, FND@mPDA-DOX-TPGS, and FND@PDA-DOX-TPGS, respectively (a: 24-hour culture, b: 48-hour culture).

### DETAILED DESCRIPTION

### Example 1: Preparation and Characterization of Fluorescent Nanodiamond Coated with Porous Polydopamine

### 1-1. Experimental Materials

Fluorescent nanodiamonds (FNDs) with sizes of 50 nm and 80 nm were purchased from Columbus NanoWorks and Addmas Nanotechnologies. Dopamine (DA) hydrochloride, TRIS, F127, 1,3,5-trimethyl benzene (TMB), ethanol (EtOH), TPGS, ammonium hydroxide solution (NH₄OH, 25%), glycidol, N-hydroxysulfosuccinimide sodium salt (sulfo-NHS), N-(3-dimethylaminopropyl)-N′-ethylcarbodiimide hydrochloride (EDC), poly-L-lysine solution (0.1% w/v in H₂O), Tween-20, potassium hydroxide (KOH), and Dulbecco's phosphate-buffered saline (DPBS) were obtained from Sigma-Aldrich. mPEG-SH (2 kDa) was purchased from NANOCS, and CellTiter-Glo® assay reagent was purchased from Promega. 4% Paraformaldehyde was purchased from Bioseasang. Doxorubicin (DOX) was purchased from Fisherbioreagents. Cytofix/Cytoperm™ Fixation/Permeabilization Kit was purchased from BD, annexin V-FITC, and propidium iodide (PI)-included dead cell apoptosis kit were purchased from BD Pharmingen™. Flash Phalloidin Green 488 was purchased from BioLegend. DAPI, bovine serum albumin (BSA), and streptavidin were purchased from Invitrogen. Phosphate-buffered saline (PBS) was purchased from Welgene. All oligonucleotides used for miRNA detection were purchased from Integrated DNA Technology, and SplintR ligase was purchased from New England Biolabs. Salmon sperm DNA and RNAsecure were purchased from ThermoFisher. HEPES buffer (1 M, pH 7.4) and fetal bovine serum were purchased from Gibco. Deionized water (DI) with a resistivity of 18.2 MΩ·cm was obtained using a Milli-Q purification system.

1-2. Preparation of Fluorescent Nanodiamond Coated with Porous Polydopamine

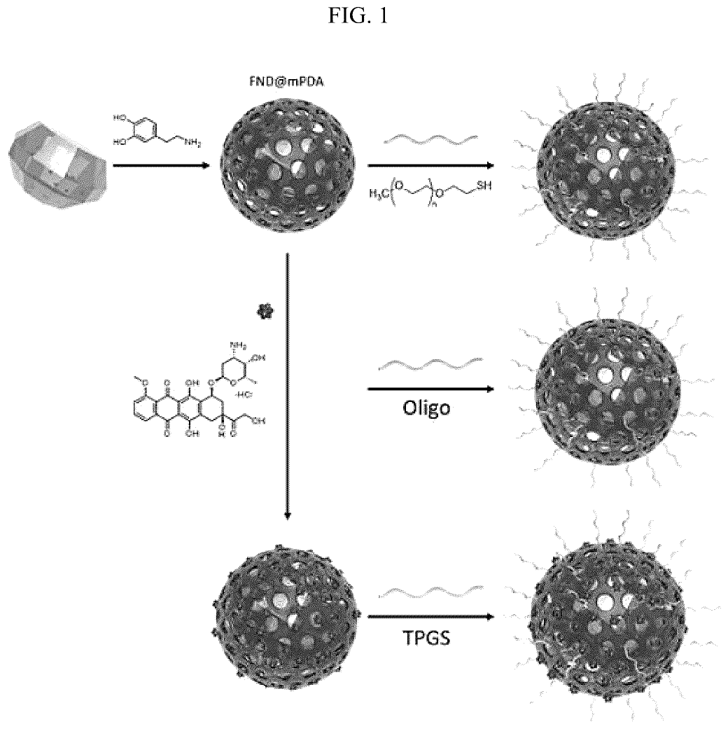

The synthesis process of a fluorescent nanodiamond (hereinafter, FND@mPDA) coated with mesoporous polydopamine (hereinafter, mPDA), and fluorescent nanodiamond surface-modified with thiol terminated methoxy polyethylene glycol (mPEG-SH), hyperbranched polyglycerol (HPG), or d-α-tocopheryl polyethylene glycol 1000 succinate (TPGS) on the FND@mPDA is shown in FIG. 1.

To prepare FND@mPDA, 0.36 g of triblock copolymer pluronic F127 (F127) and 0.63 mL of 1,3,5-trimethyl benzene (TMB) were dissolved in a mixed solution of 65 mL of deionized water and 60 mL of ethanol solution to prepare a mixture, and then the mixture was stirred at room temperature for 1 hour. Then, 10 mL of distilled water containing 90 mg of dopamine (DA) and FND (Table 1) at various concentrations and TRIS was added to the mixture, respectively, and stirred at room temperature (25° C.) for 24 hours.

TABLE 1

| | | | mPDA shell |
|---|---|---|---|
| Condition | Dopamine (mM) | FND (mg) | thickness (nm) |
| A | 2.53 | 10 | 25.5 ± 3.6 |
| B | 2.53 | 20 | 16.3 ± 2.4 |
| C | 2.53 | 40 | 7.6 ± 1.8 |
| D | 0.84 | 10 | 3.9 ± 1.4 |
| E | 1.69 | 10 | 13.6 ± 2.0 |
| F | 2.53 | 10 | 25.5 ± 3.6 |

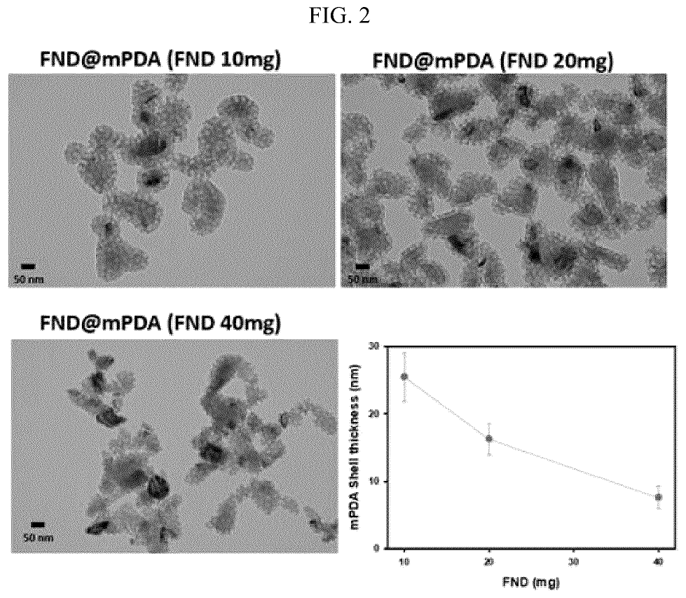

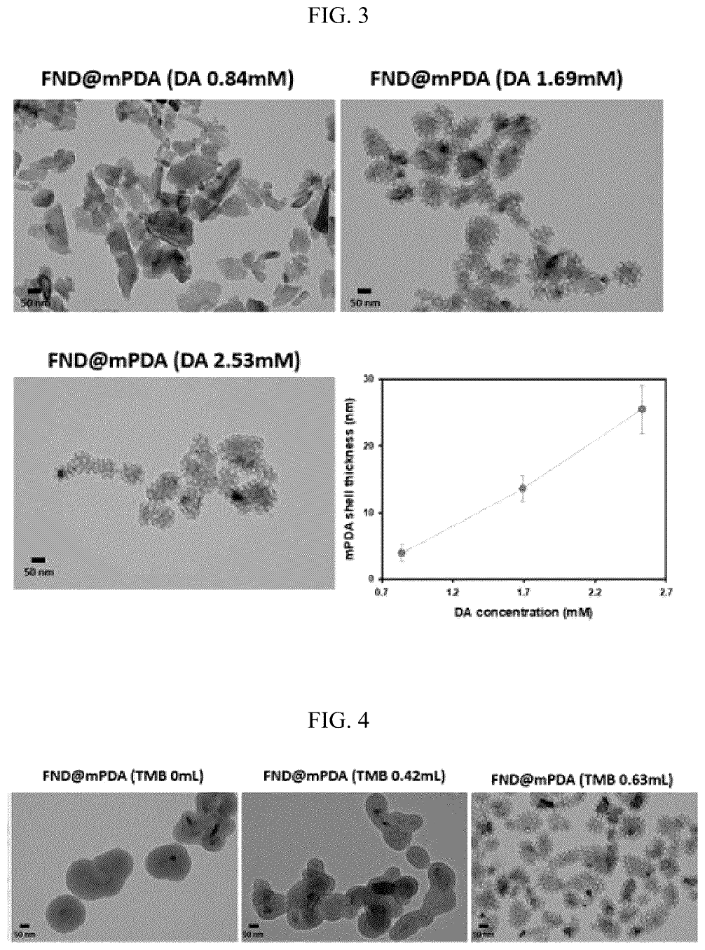

The mPDA-coated FND was separated by centrifugation at 27,670 g (15,000 rpm) for 10 minutes, and the precipitated FND@mPDA was redispersed in a mixed solution of ethanol and acetone (2:1 v/v) and ultrasonicated for 1 hour to remove micelles formed via F127 and TMB. The above purification step was repeated three times to obtain FND@mPDA dispersed in water. The mPDA shell thickness of FND@mPDA prepared under each condition in Table 1 decreased as the concentration of FND increased (FIG. 2) and increased as the concentration of dopamine increased (FIG. 3).

In addition, FND@mPDA was prepared by the same method as above by fixing the concentration of dopamine (2.53 mM) and changing the amount of FND and TMB added. Then, the pore size according to each condition was compared using a transmission electron microscope (TEM, JEOL 1400). The pore size was measured using a Micromeritics TriStar II Plus nitrogen adsorption-desorption isotherm system, and the preparation conditions are shown in Table 2.

TABLE 2

| Category | Dopamine (mM) | FND (mg) | TMB (mL) |
|---|---|---|---|
| 1 | 2.53 | 10 | 0 |
| 2 | 2.53 | 20 | 0.42 |
| 3 | 2.53 | 10 | 0.63 |

As a result, it was confirmed that FND@PDA with a nonporous PDA shell was generated in the absence of TMB, and the pore size of FND@mPDA increased as the concentration of TMB increased (FIG. 4). These results suggest that self-assembled micelles cannot be generated in the absence of TMB molecules due to the weak interaction between F127 molecules.

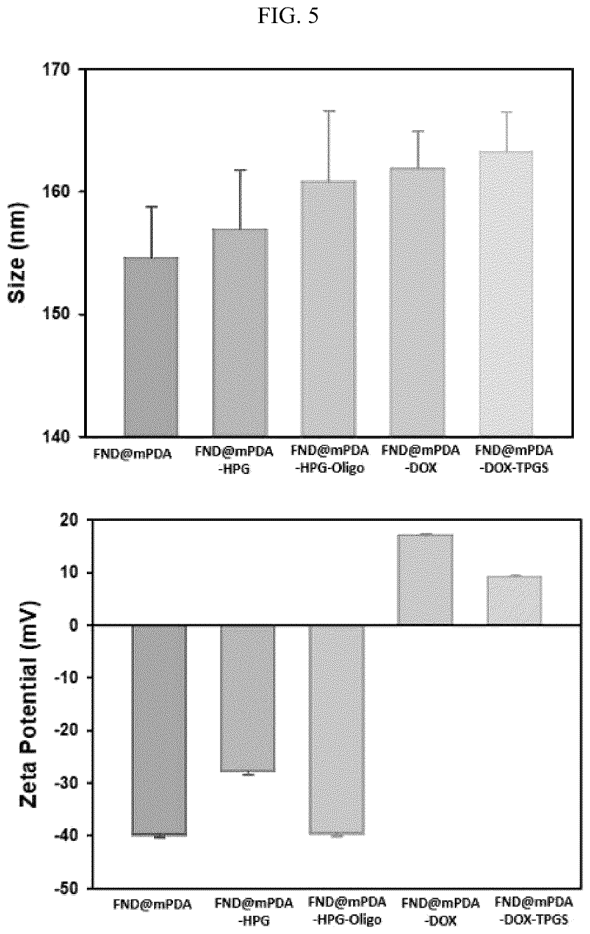

The sizes and zeta potentials of the prepared FND@mPDA, and FND@mPDA-HPG-COOH, FND@mPDA-HPG-COOH-Oligo, FND@mPDA-DOX, and FND@mPDA-DOX-TPGS described in the Examples below are shown in FIG. 5, and the size and shape of each material were measured by a JEOL 1400 TEM (120 kV accelerating voltage) equipped with an AMT XR-111 digital camera.

### 1-3. Physicochemical Characteristics of FND@mPDA

To determine the physicochemical properties of FND@mPDA, thermogravimetric analysis (TGA) and nitrogen adsorption analysis were performed.

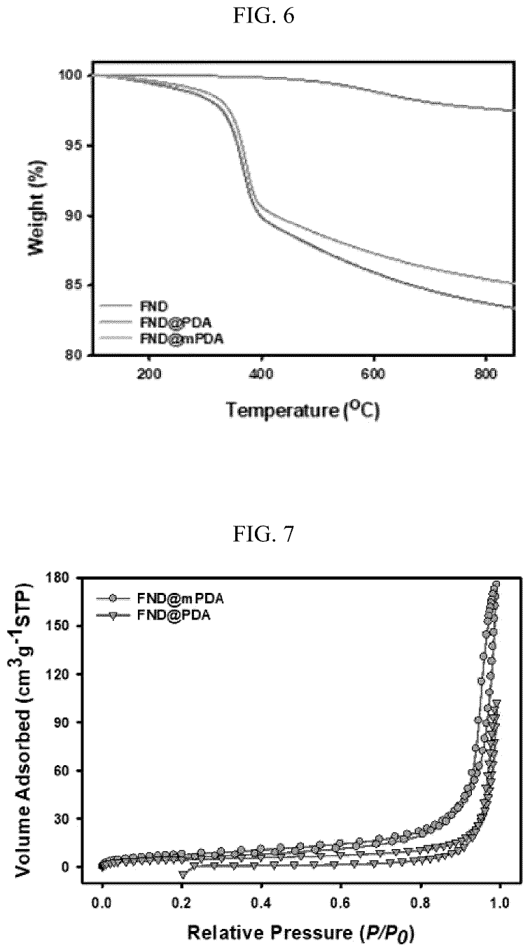

The weight loss of FND, FND@PDA, and FND@mPDA was measured by an SDT Q600 TGA (TA Instruments), and the TGA curves of FND, FND@PDA, and FND@mPDA were analyzed under a nitrogen environment. As a result, it was confirmed that the weight loss of each material was 2.5% for FND, 16.7% for FND@PDA, and 14.9% for FND@mPDA, and in particular, FND@mPDA showed a lower weight loss than FND@PDA because the density of the polydopamine shell is lower than that of FND@PDA due to the porous structure (FIG. 6).

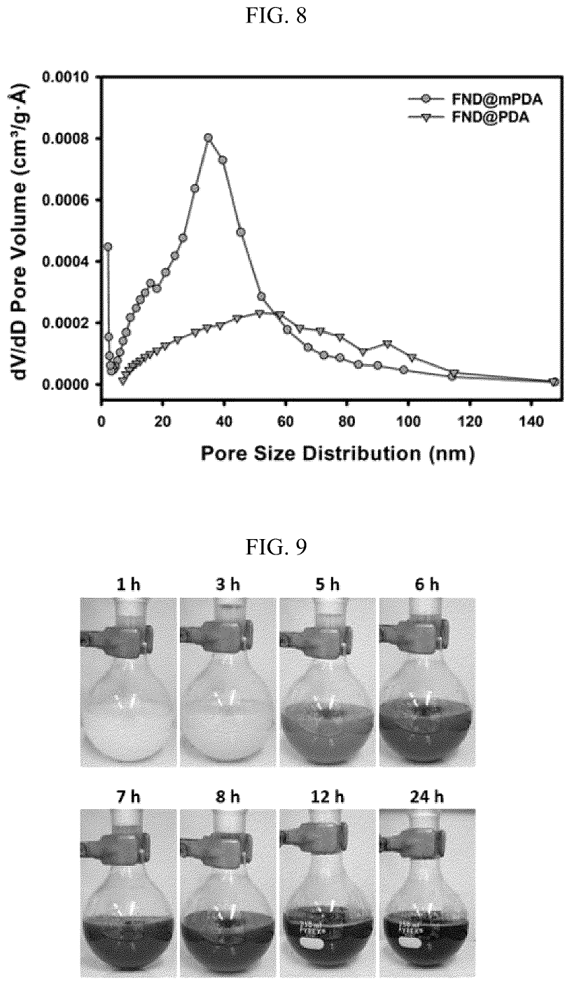

The nitrogen adsorption-desorption isotherm of FND@mPDA shows a typical type IV curve with a distinct hysteresis loop within the P/PO range of 0.4 to 0.9 according to the IUPAC nomenclature (FIG. 7), indicating a wider pore size distribution of 20 to 60 nm compared to FND@PDA (FIG. 8).

As a result of measuring the surface area and pore volume of FND@mPDA and FND@PDA, FND@mPDA had a surface area of 31.4 m²/g and a pore volume of 0.27 cm³/g, while FND@PDA had a surface area of 16.8 m²/g and a pore volume of 0.16 cm³/g. It was confirmed that the surface area and pore size increased more when mesoporous polydopamine was coated than polydopamine, and this difference means that FND@mPDA can load a larger amount of drug than FND@PDA, thereby increasing the drug delivery efficiency.

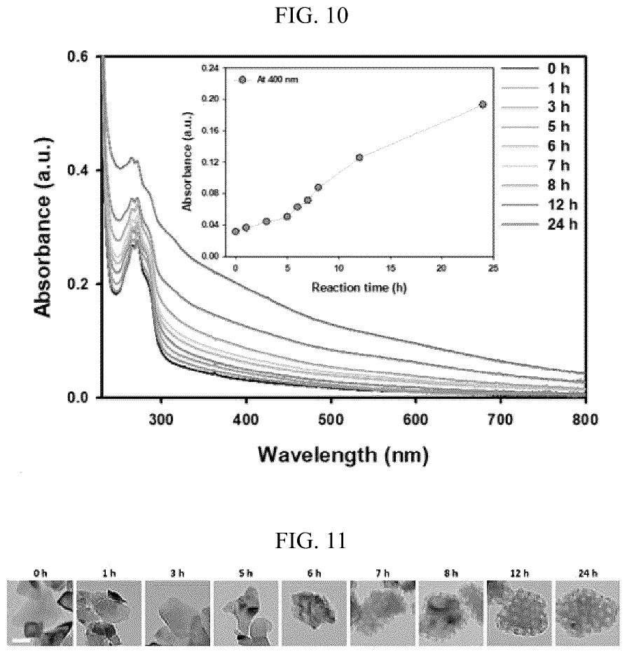

In addition, the kinetics of mPDA shell formation were analyzed by photography, UV-vis absorption, and TEM to investigate the reaction of mPDA on the FND surface. It was confirmed with the naked eye that when FND@mPDA was prepared by the method of Example 1-2, the color of the mixture darkened as the reaction time elapsed (FIG. 9), indicating that the F127 and TMB molecules formed micelles through self-assembly and then formed complex micelles with the oxidized and self-polymerized polydopamine monomers. It was found that as the reaction time elapsed, an mPDA shell was formed on the FND surface, and the UV absorption spectra increased (FIG. 10). FIG. 11 shows the results of observing mPDA formed on the FND surface as the reaction time elapsed using a TEM.

In addition, the physicochemical properties of FND and FND@mPDA were compared by transmission electron microscopy (TEM), UV-vis absorption, and photoluminescence (PL) spectroscopy.

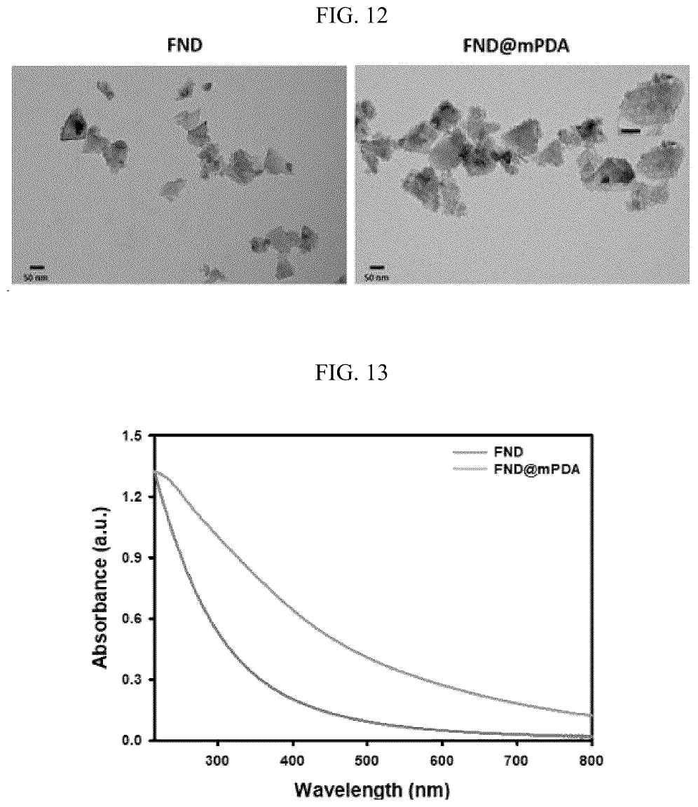

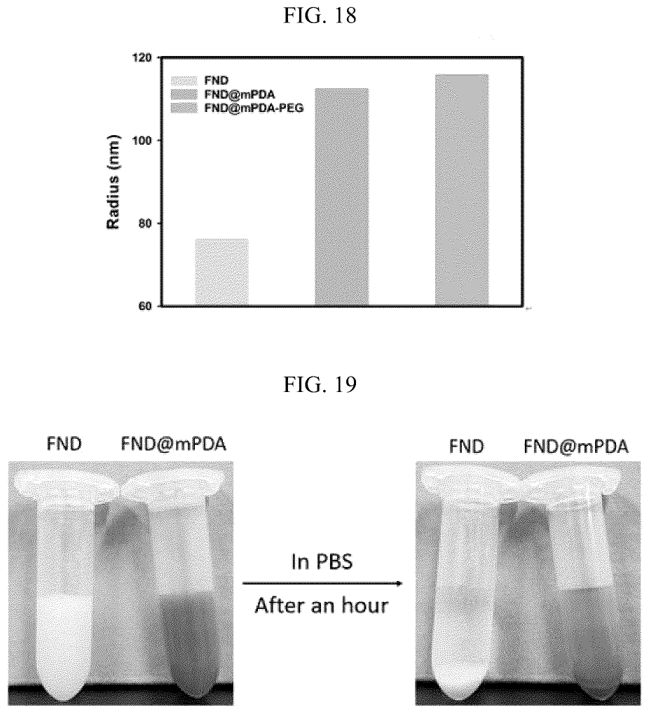

The TEM image observation results showed that FND had an irregular shape, sharp edges, and a wide size distribution, while FND@mPDA had smooth edges and an increased diameter of 225 nm compared to FND (FIGS. 12 and 18).

UV-vis absorption spectra and photoluminescence (PL) spectra were observed using a Tecan Spark 10 M Multi-Mode Microplate Reader, and the UV-vis absorption analysis results showed that FND@mPDA exhibited significantly higher light absorption characteristics than FND (FIG. 13). The emission photons and PL efficiencies of FND@mPDA and FND prepared under conditions D and E of Example 1-2 were compared using a total internal reflection fluorescence (TIRF) microscope. As a result, the average emission photons of FND@mPDA were lower than those of FND, confirming that the fluorescent nanodiamond coated with mesoporous polydopamine has different physicochemical properties compared to before coating (Table 3).

In addition, the pores present in the mesoporous polydopamine can make the mesoporous polydopamine shell surrounding the fluorescent nanodiamond less dense, which can absorb less excitation/emission light compared to polydopamine, thereby increasing the fluorescence intensity.

TABLE 3

| | Average emitted photons |
|---|---|
| Category | (PL efficiency) |
| FND | 7141 (100%), N = 832 |
| FND@ mPDA (D condition, | 5498 (77%), N = 863 |
| shell thickness 3.9 nm) | |
| FND@ mPDA (E condition, | 5040 (71%), N = 665 |
| shell thickness 13.6 nm) | |

Additionally, an experiment was conducted to confirm whether FND@mPDA has the property of absorbing light and generating heat. If such a property exists, it is possible to kill cancer cells by the heat generated by irradiating light on FND@mPDA injected into cancer cells.

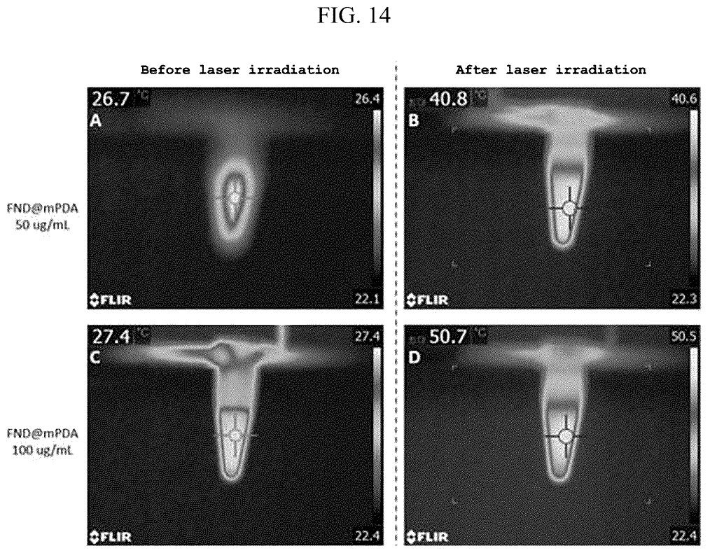

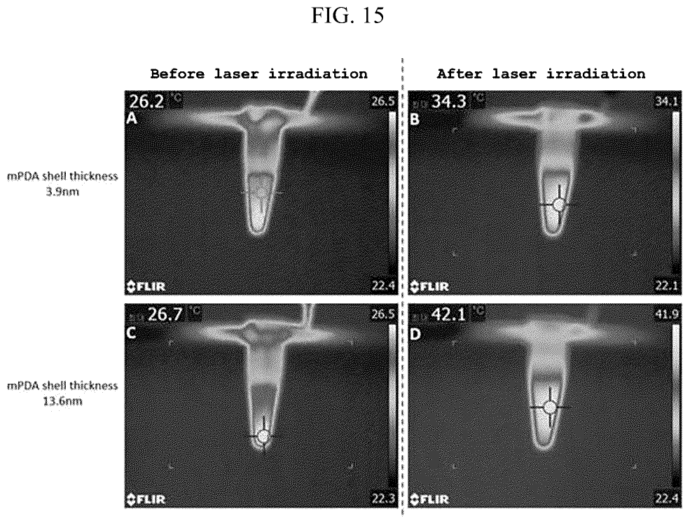

When the heat generation was confirmed by irradiating the FND@mPDA with an 808 nm laser, it was confirmed that the heat generation increased as the concentration of the FND@mPDA increased when 50 μg/mL or 100 μg/mL of FND@mPDA was irradiated with light (FIG. 14). It was confirmed that the heat generation increased as the mPDA thickness increased when 50 μg/mL FND@mPDA with an mPDA shell thickness of 3.9 nm or 13.6 nm was irradiated with light (FIG. 15).

### 1-4. Evaluation of Drug Delivery Efficiency of FND@mPDA

Since FND@mPDA can deliver drugs due to its large surface area, we analyzed whether drug delivery was possible by loading doxorubicin.

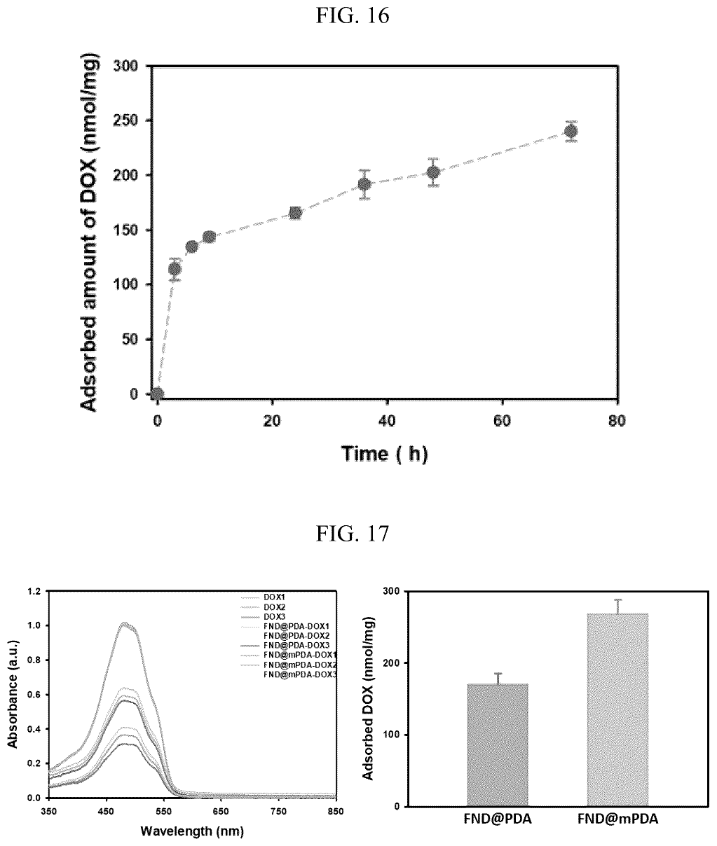

It was confirmed that FND@mPDA and doxorubicin (DOX) were mixed in HEPES buffer (25 mM, pH 7.4) to prepare FND@mPDA-DOX, and the size and surface charge changed due to the DOX absorbed on the mPDA shell surface (FIG. 5). To determine the optimal mixing time, 86 μM of DOX and 0.2 mg of FND@mPDA were mixed in 1 mL of HEPES buffer to investigate the adsorption kinetics of DOX. The DOX-adsorbed FND@mPDA was precipitated by centrifugation, and the absorbance of the supernatant was measured by UV-Vis absorption for up to 72 hours. The results showed that 113.95 nmol of DOX per 1 mg of FND@mPDA was rapidly loaded within 3 hours, and then the loading amount slowly increased (FIG. 16).

Then, the drug loading efficiency between FND@PDA and FND@mPDA was compared by UV-Vis absorption, and after centrifuging FND@PDA-DOX or FND@mPDA-DOX, the absorbance of the supernatant was measured at a wavelength of 482 nm to calculate the DOX adsorbed on the PDA or mPDA shell surface (using a UV-2600 (Shimadzu) UV-Vis spectrophotometer).

As a result, 268 nmol of DOX was loaded on FND@mPDA, while a 37% reduced amount (170 nmol) of DOX was loaded on FND@PDA-DOX, confirming that the drug loading and delivery efficiency were significantly superior when fluorescent nanodiamonds were coated with mesoporous dopamine compared to when coated with dopamine (FIG. 17).

### Example 2: Preparation and Characterization of Fluorescent Nanodiamonds Surface-Modified with mPEG-SH and Coated with Porous Polydopamine

2-1. Preparation of FND@mPDA Surface-Modified with mPEG-SH

To enhance the colloidal stability of FND@mPDA, the surface of FND@mPDA was modified with mPEG-SH using a Michael addition reaction. Specifically, 4 mg of mPEG-SH (2 kDa) and 2 μL of NH₄OH were added to 10 mL of an aqueous FND@mPDA solution at a concentration of 0.02 mg/mL prepared in Example 1-2 to prepare a mixture, and then the mixture was stirred at room temperature (25° C.) for 16 hours. The mixture was then centrifuged at 27,670 g (15,000 rpm) for 15 minutes, and the precipitated FND@mPDA surface-modified with mPEG-SH was redispersed in water. The above purification step was repeated twice to obtain FND@mPDA surface-modified with mPEG-SH (FND@mPDA-PEG).

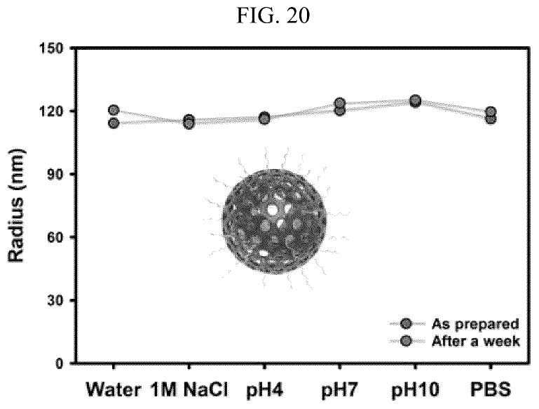

It was confirmed that FND@mPDA-PEG showed an increased particle size compared to FND and FND@mPDA (FIG. 18). The colloidal stability of FND@mPDA-PEG was evaluated by dynamic light scattering (DLS) under a wide range of pH and high salt conditions. As a result, it was confirmed that FND and FND@mPDA were precipitated within 1 hour in PBS buffer (FIG. 19), whereas FND@mPDA-PEG maintained the same hydrodynamic radius before and one week after treatment in 1 M NaCl aqueous solution, pH 4, pH 7, and pH 10 solutions, whose pH was adjusted using hydrochloric acid and sodium hydroxide in PBS buffer, and PBS buffer (DynoPro Nanostar, Waytt), indicating excellent colloidal stability (FIG. 20).

2-2. Biocompatibility Analysis of FND@mPDA Surface-Modified with mPEG-SH

To apply nanomaterials in the biomedical field, biocompatibility need to be guaranteed. Therefore, the biocompatibility of FND@mPDA surface-modified with mPEG-SH (FND@mPDA-PEG) was analyzed to evaluate its effects on cell proliferation and toxicity in HeLa cells.

Specifically, cell viability was evaluated under various treatment conditions using the CellTiter-Glo® luminescent cell viability assay according to the manufacturer's instructions. HeLa cells (0.6×10⁴ cells/well) were cultured in 96-well tissue culture plates containing 100 μL/well culture medium, and then treated in triplicate with 50 μg/mL, 100 μg/mL, 150 μg/mL, or 200 μg/mL of FND or FND@mPDA-PEG and incubated at 37° C. for 12 hours. After equilibrating the cells at room temperature for 30 minutes, CellTiter-Glo® reagent was added in a volume equal to that of the cell culture medium, and then shaken on an orbital shaker for 2 minutes to induce cell lysis. The plates were incubated at room temperature for 10 minutes to stabilize the luminescence signal, and the luminescence was read using a 2103 EnVision Multilabel Plate Reader.

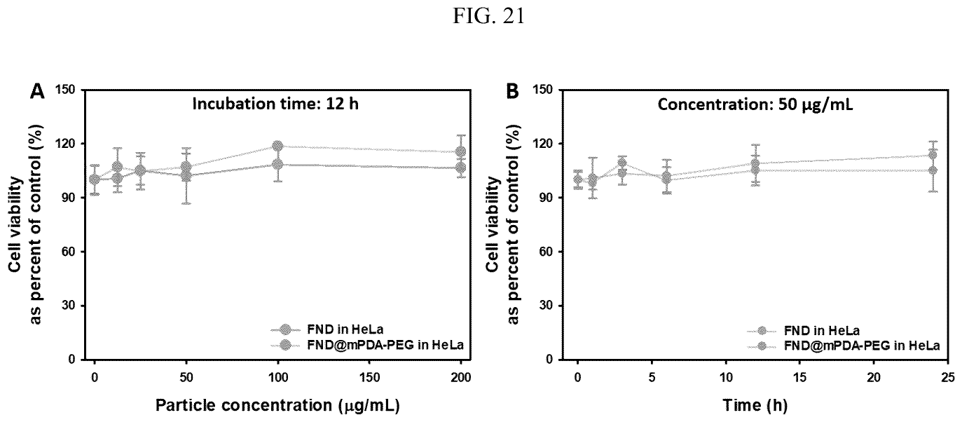

As a result, it was confirmed that after 12 hours of culture, the survival rate of cells treated with FND@mPDA-PEG was higher than that of cells treated with FND, regardless of concentration (FIG. 21A), and when cells were treated with 50 μg/mL of FND and FND@mPDA-PEG, respectively, the cell viability was not significantly affected over time, indicating high biological stability (FIG. 21B).

In addition, analysis was performed using FACS (BD Accuri™, BD Biosciences) to evaluate the biocompatibility of FND@mPDA-PEG. Specifically, HeLa cells were incubated with 50 μg/mL of FND, FND@mPDA-PEG, or doxorubicin for the control experiment at 37° C. for 16 hours and washed with PBS. Cells (1×10⁵ cells/sample) were suspended in 100 μL of 1× binding buffer and stained with 5 μL of Annexin V-FITC and 5 μL of PI. The cells were gently vortexed and incubated at room temperature for 15 minutes in the dark. 400 μL of 1× binding buffer was added to each tube and then analyzed by flow cytometry. 10,000 or more events were collected and the data were analyzed using FlowJo™ version 10.7 software.

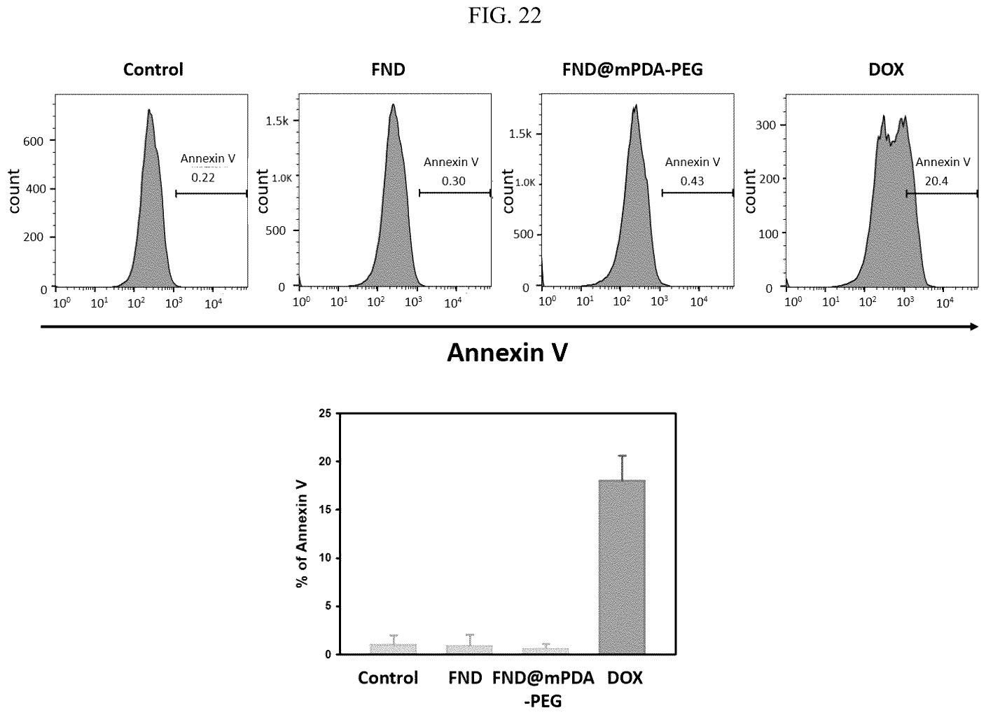

As a result, it was confirmed that doxorubicin induced apoptosis of HeLa cells, whereas treatment with FND or FND@mPDA-PEG did not affect cell survival during culture (FIG. 22).

2-3. Evaluation of Cell Imaging (Lesion Labeling) Using FND@mPDA Surface-Modified with mPEG-SH

An experiment was performed to evaluate cell imaging using FND@mPDA-PEG. Specifically, HeLa cells were incubated with 25 μg/mL of FND or FND@mPDA-PEG at 37° C. for 16 hours and washed with PBS. Then, the cells were resuspended in a fixation/permeabilization solution (BD, Cytofix/Cytoperm™ Fixation/Permeabilization Kit) at 4° C. for 20 minutes and washed twice with 1×BD Perm/Wash™ buffer. The cells were then blocked with 5% fetal bovine serum in PBS at 37° C. for 30 minutes and washed twice with PBS. In a dark room blocked from light, the cells were stained with Flash Phalloidin Green 488 (Biolegend) 1:50 in PBS at 37° C. for 30 minutes and then washed twice with PBS. The samples were mounted on DAPI (Invitrogen) and ProLong Gold anti-fade mounts, and analyzed with a Zeiss LSM 880 confocal laser scanning microscope.

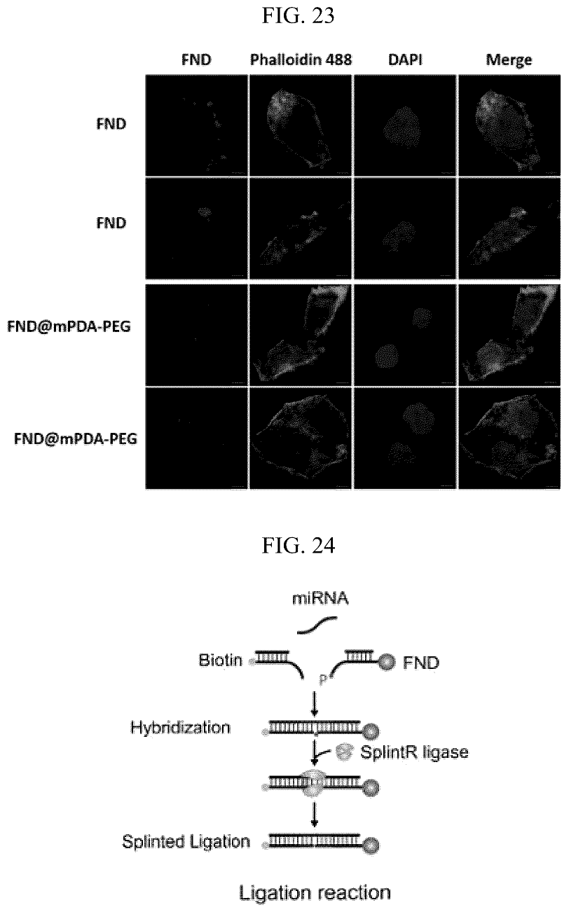

As a result, it was confirmed that when FND@mPDA-PEG was treated, which was well dispersed due to high colloidal stability, most of it was internalized into cancer cells, showing excellent cancer cell labeling, whereas when FND was treated, aggregation occurred due to low colloidal stability, and the aggregated particles showed nonspecific adhesion to the cell membrane, making it unsuitable for cancer cell labeling (FIG. 23).

Thus, it was confirmed that surface modification of FND@mPDA with mPEG-SH increased colloidal stability, enabling excellent cell imaging.

### Example 3: Preparation and Characterization of Fluorescent Nanodiamonds Surface-Modified with Hyperbranched Polyglycerol and Coated with Porous Polydopamine

3-1. Preparation of FND@mPDA Surface-Modified with Hyperbranched Polyglycerol

FND@mPDA surface-modified with hyperbranched polyglycerol (hereinafter, HPG) was prepared.

Specifically, 10 mg of FND@mPDA was centrifuged at 17,910 g (15,000 rpm) and the supernatant was removed. The separated FND@mPDA was dispersed in glycidol and washed twice for solution replacement. A 2.5 mg/mL suspension of FND@mPDA in 4 mL of glycidol was ultrasonicated and stirred at 90° C. for 1 hour under a nitrogen environment. To the mixture was added 100 mg of succinic anhydride, and the mixture was stirred again at 90° C. for 1 hour under a nitrogen environment. The prepared FND@mPDA-HPG-COOH was purified by centrifugation at 23,005 g (17,000 rpm) for 10 minutes, and washed twice with deionized water to remove unreacted polyglycerol and succinic anhydride.

Additionally, FND@mPDA-HPG-Oligo was prepared by conjugating oligonucleotides to the prepared FND@mPDA-HPG-COOH.

Specifically, 12 mg of sulfo-NHS and 16 mg of EDC were introduced into a 10 mg/mL suspension of FND@mPDA-HPG-COOH dissolved in 1 mL of water for 30 minutes to activate the surface carboxylation group, and then the particles were purified through centrifugation (15,000 rpm, 10 minutes). The separated particles were redispersed in water, and the above process was repeated twice. 50 μL of Oligo-NH₂ at a concentration of 100 μM was mixed with 10 mg of activated FND@mPDA-HPG-COOH (in 1 mL water), stirred at room temperature for 2 hours, and then centrifuged at 15,000 rpm to prepare FND@mPDA-HPG-Oligo.

3-2. Evaluation of miRNA Detection by FND@mPDA Surface-Modified with Hyperbranched Polyglycerol

An experiment was performed to determine whether FND@mPDA surface-modified with hyperbranched polyglycerol could detect miRNA as a fluorescent probe.

Specifically, the 5′-terminal of Oligo-NH₂ used for the preparation of FND@mPDA-HPG-Oligo was phosphorylated to be anti-complementary to miRNA, and the 3′-terminal was modified with an amine to label FND@mPDA-HPG-COOH. T20 was inserted as a spacer, and the Oligo-NH₂ and FND@mPDA-HPG-COOH were conjugated according to the method of Example 3-1 to prepare an FND@mPDA-HPG-Oligo probe (FND probe).

Probe biotin was biotinylated at the 5′-terminal to immobilize the surface through streptavidin-biotin interaction and had a different anti-complementarity to miRNA. The hybridization process through the binding reaction of FND@mPDA-HPG-Oligo and miRNA is shown in FIG. 24.

Probe_stem 1 and 2 were designed to increase the base stacking effect related to hybridization stability when miRNA binds to complementary substances. The target miRNA (miR-125b) was designed to have the base sequence of SEQ ID NO: 1, and all probe solutions except the FND probe were prepared at the same ratio. To label miRNA using SplintR, 1 μL of synthesized miRNA (20 nM, 200 nM), 0.8 μL of FND probe at a concentration of 10 mg/mL, 1 μL of probe solution at a concentration of 100 nM, and 0.5 μL of SplintR ligase were mixed to make a total volume of 10 μL. The mixture was incubated at 37° C. for 2 hours, and then centrifuged at 17,910 g (15,000 rpm) for 5 minutes to increase miRNA detection efficiency. After removing the supernatant, the pellet was diluted with 100 μL of deionized water containing 0.01% tween and 0.1 mg/mL BSA (TB0.1) and injected into a fluid sample chamber of a single molecule counting (SMC) setup. The SMC setup used a prism-type total internal reflection fluorescence microscope equipped with an EMCCD camera (iXon DV897ECS-BV, Andor Technology, UK). The binding buffer was composed of 50 mM Tris-HCl (pH 8.0), 10 mM MgCl₂, 100 μM ATP, 10 mM DTT, and 0.1 mg/mL sheared salmon sperm DNA. All buffers used for binding and dilution were treated with RNAsecure or used with RNase-free water.

The base sequences of the target miRNA and the probes used are shown in Table 4.

TABLE 4

| | | Base sequence | |
|---|---|---|---|
| Category | Name | (5′ → 3′) | SEQ ID NO: |
| Targe miRNA | miR-125b | ucc cug aga ccc | 1 |
| | | uaa cuu gug a | |
| probe_Bio | P5_bio_125b | 5′Biotin-GAA GTC | 2 |
| | | TCG ACG GAC TCA | |
| | | CAA GTT | |
| probe_Phos | P6_phos_125b_FND | 5′Phos-AGG GTC TCA | 3 |
| | | GGG AGT TGT CGA | |
| | | TTT GGT CCA TTT | |
| | | TTT TTT TTT TTT | |
| | | TTT TT-Amine | |
| probe_stem1 | P5_T0 | TTT TTG TCC GTC | 4 |
| | | GAG ACT TC | |
| probe_stem2 | P6_T0 | TGG ACC AAA TCG | 5 |
| | | ACA ACT TTT T | |

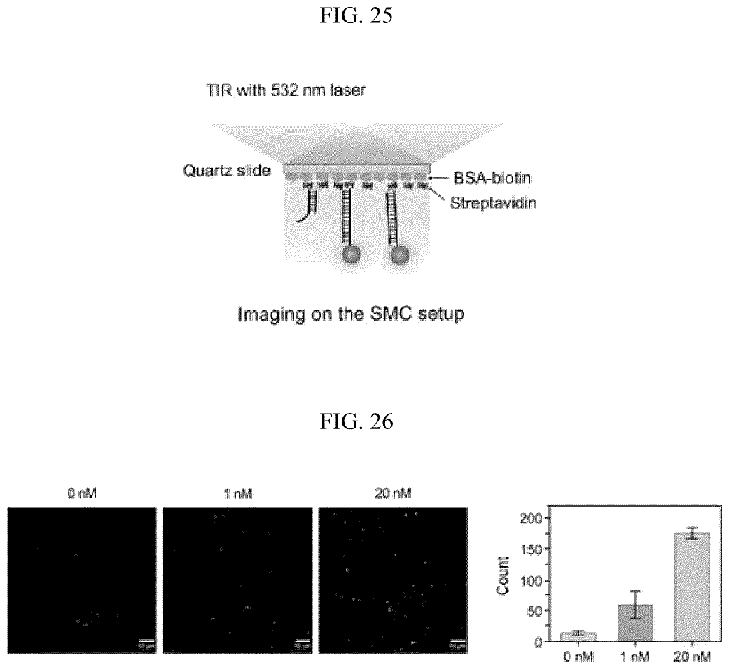

Then, a coverslip and a quartz slide containing perforated holes were prepared, and the drill holes in the quartz slide were designed to serve as the inlet and outlet. They were sequentially washed by ultrasonication with acetone and 1 M KOH for 20 minutes, respectively, and the coverslips and quartz slides were assembled. Each sample was injected through the inlet and outlet of the fluid sample chamber equipped with a syringe pump system. The SMC TIRF microscopy-based setup for quantifying miRNA is shown in FIG. 25.

As a result of detecting the fluorescence signal when the miRNA concentration was 0, 1, and 20 nM, respectively, the average number of fluorescent spots detected was 13, which was very small in the case of 0 nM (control), and when the miRNA was 1 nM, the number of fluorescent spots detected was about 60, which was a 4.5-fold increase compared to the control, and when the miRNA was 20 nM, the number of fluorescent spots detected was 175, which was a 13.5-fold increase compared to the control group and a 2.9-fold increase compared to 1 nM (FIG. 26).

These results indicate that when detecting miRNA using the FND probe prepared with FND@mPDA surface-modified with hyperbranched polyglycerol, it selectively acts only when miRNA is present, and the fluorescence spot of the detected molecule increases as the concentration of miRNA increases, confirming that FND@mPDA surface-modified with hyperbranched polyglycerol can be directly used as a fluorescent probe for miRNA detection.

### Example 4: Preparation and Characterization of Fluorescent Nanodiamond Surface-Modified with d-α-Tocopheryl Polyethylene Glycol 1000 Succinate and Coated with Porous Polydopamine

4-1. Preparation of FND@mPDA Surface-Modified with d-α-Tocopheryl Polyethylene Glycol 1000 Succinate

1 mg of FND@PDA or FND@mPDA was dispersed in 950 μL of 25 mM HEPES buffer pH 7.4, and then mixed with 0.86 M doxorubicin. The mixture was stirred at room temperature for 72 hours, and then centrifuged at 17,920 g (15,000 rpm) for 10 minutes to obtain doxorubicin (DOX)-loaded FND@PDA or FND@mPDA, which was then redispersed in HEPES buffer (25 mM, pH 7.4). The above purification process was repeated twice to remove the excess doxorubicin.

1 mg of doxorubicin-loaded FND@PDA or FND@mPDA dispersed in 950 μL of HEPES buffer (25 mM, pH 7.4) was mixed with 50 μL of TPGS (1 mg/mL), stirred at room temperature for 24 hours, and centrifuged at 17,920 g (15,000 rpm) for purification to prepare doxorubicin-loaded FND@PDA (FND@PDA-DOX-TPGS) or FND@mPDA (FND@mPDA-DOX-TPGS) surface-modified with TPGS.

### 4-2. Evaluation of Cellular Uptake of FND@mPDA-DOX-TPGS

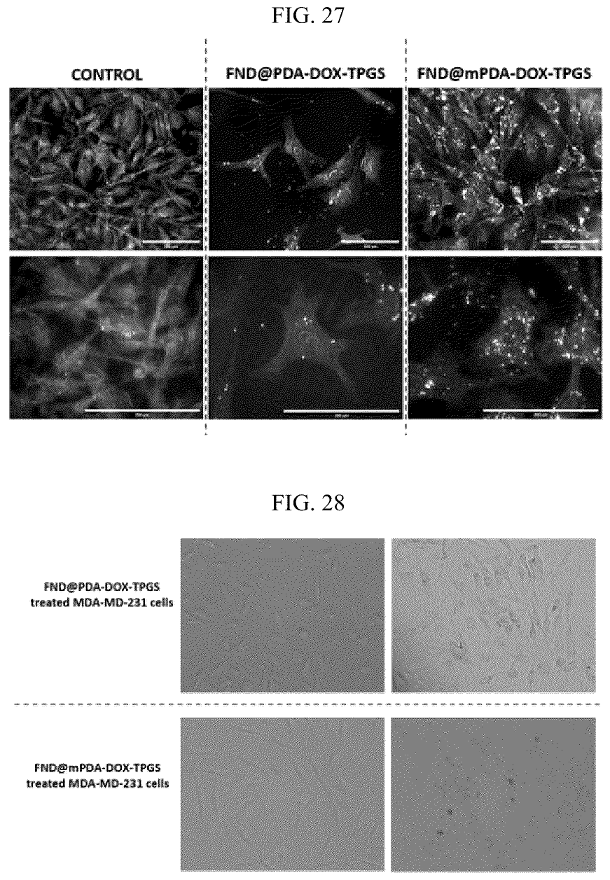

The uptake of FND@PDA-DOX-TPGS or FND@mPDA-DOX-TPGS in MDA-MB-231 cells was observed using a reflection dark-field microscope (epi-illumination, Thorlabs, Cerna system). The cells were seeded in a confocal dish at a density of 5×10⁴ and cultured for 24 hours, then treated with 25 μg/mL of FND@PDA-DOX-TPGS or FND@mPDA-DOX-TPGS and cultured at 37° C. for 24 hours. Then, they were washed with DPBS, fixed with 4% PFA for 1.5 hours, followed by twice with DPBS. The fixed cells were supplemented with 1 mL of PBS and visualized using a reflection dark-field microscope to confirm a cell structure (FIG. 27). MDA-MB-231 cells that were not treated with FND@PDA-DOX-TPGS or FND@mPDA-DOX-TPGS were used as a control. Compared with the control, FND@mPDA-DOX-TPGS was easily observed in the treated cells without nonspecific interactions with the cell membrane. When the cellular uptake was measured by bright-field imaging, no significant difference was observed between FND@PDA-DOX-TPGS and FND@mPDA-DOX-TPGS (FIG. 28).

### 4-3. Evaluation of Drug Delivery Efficiency of FND@mPDA-DOX-TPGS

To compare the drug delivery efficiency of FND@PDA-DOX-TPGS and FND@mPDA-DOX-TPGS, a cell viability test was performed, and the CellTiter-Glo® luminescent cell viability assay was performed according to the manufacturer's instructions to confirm cell viability.

Specifically, MDA-MD-231 cells (0.6×104 cells/well) were cultured in 96-well tissue culture plates with 100 μL/well of culture medium. The cells were treated three times with 0, 5, or 10 μM DOX; FND@PDA-DOX-TPGS loaded with 0, 5, or 10 μM DOX; and FND@mPDA-DOX-TPGS loaded with 0, 5, or 10 μM DOX, respectively, and incubated at 37° C. After 24 or 48 hours, the cells were allowed to equilibrate at room temperature for approximately 30 minutes before adding the CellTiter-Glo® reagent. CellTiter-Glo® reagent, at the same concentration as the cell culture medium, was added to each well and then shaken on an orbital shaker for 2 minutes to induce cell lysis. The plates were incubated at room temperature for 10 minutes to stabilize the luminescence signal, and the luminescence was read using a 2103 EnVision Multilabel Plate Reader.

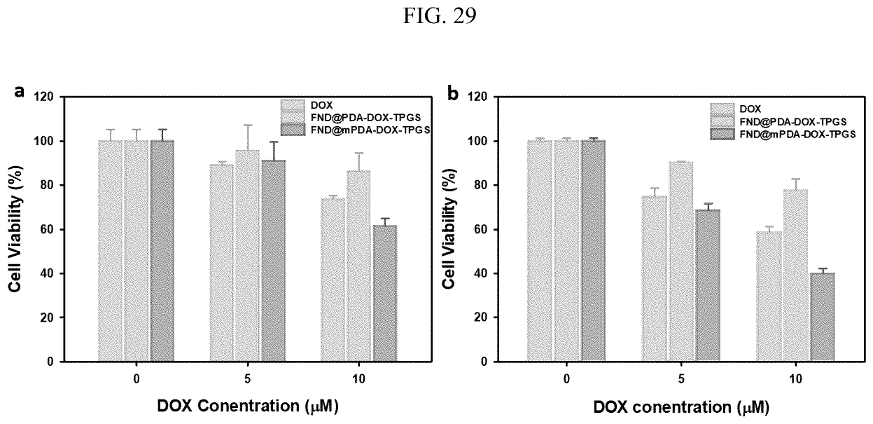

As a result, it was confirmed that FND@mPDA-DOX-TPGS loaded with DOX showed a higher cytotoxic effect than when cells were treated with DOX or FND@PDA-DOX-TPGS (FIG. 29, a: 24-hour incubation, b: 48-hour incubation).

It was confirmed from these results that FND@mPDA-DOX-TPGS had increased drug absorption efficiency and decreased multi-drug resistance (MDR) compared to FND@PDA-DOX-TPGS.

From the above description, those skilled in the art to which the present disclosure pertains will understand that the present disclosure can be implemented in other specific forms without changing its technical idea or essential features. In this regard, it should be understood that the embodiments described above are exemplary in all respects and are not intended to be limiting. The scope of the present disclosure should be interpreted that all changes or modifications derived from the meaning and scope of patent claims to be described below rather than the detailed description above and their equivalent concepts are included within the scope of the present disclosure.

1. A fluorescent nanodiamond coated with porous polydopamine.

2. The fluorescent nanodiamond of claim 1, wherein the fluorescent nanodiamond is surface-modified with poly(ethylene glycol) methyl ether thiol (mPEG-SH), hyperbranched polyglycerol, or d-α-tocopheryl polyethylene glycol 1000 succinate.

3. The fluorescent nanodiamond of claim 1, wherein the fluorescent nanodiamond has a thickness of 1 nm to 30 nm.

4. The fluorescent nanodiamond of claim 1, wherein the fluorescent nanodiamond coated with porous polydopamine has increased drug delivery efficiency compared to fluorescent nanodiamond that are not coated with porous polydopamine or fluorescent nanodiamond coated with non-porous polydopamine.

5. A method for preparing a fluorescent nanodiamond coated with porous polydopamine, comprising:
(a) preparing a mixture by mixing triblock copolymer pluronic F127 and 1,3,5-trimethyl benzene (TMB);
(b) adding dopamine, fluorescent nanodiamond, and TRIS to the mixture;
(c) ultrasonicating a precipitate obtained by centrifuging the mixture after the addition in step (b); and
(d) redispersing the precipitate in a mixed solution of ethanol and acetone after the ultrasonication in step (c) to obtain a fluorescent nanodiamond coated with porous polydopamine.

6. The method of claim 5, wherein the concentration of the dopamine is 1 mM to 3 mM.

7. The method of claim 5, wherein the content of the 1,3,5-trimethyl benzene is 0.1 mL to 1 mL.

8. A composition for thermotherapy, comprising the fluorescent nanodiamond coated with porous polydopamine of claim 1 as an active ingredient.

9. The composition of claim 8, wherein the fluorescent nanodiamond is surface-modified with mPEG-SH, hyperbranched polyglycerol, or d-α-tocopheryl polyethylene glycol 1000 succinate.

10. The composition of claim 8, wherein the composition is for cancer treatment.

11. A composition for drug delivery, comprising the fluorescent nanodiamond coated with porous polydopamine of claim 1.

12. The composition of claim 11, wherein the fluorescent nanodiamond is surface-modified with mPEG-SH, hyperbranched polyglycerol, or d-α-tocopheryl polyethylene glycol 1000 succinate.

13. A composition for lesion labeling, comprising the fluorescent nanodiamond coated with porous polydopamine of claim 1.

14. The composition of claim 13, wherein the fluorescent nanodiamond is surface-modified with mPEG-SH, hyperbranched polyglycerol, or d-α-tocopheryl polyethylene glycol 1000 succinate.

15. A composition for detecting nucleic acids, comprising the fluorescent nanodiamond coated with porous polydopamine of claim 1.

16. The composition of claim 15, wherein the fluorescent nanodiamond is surface-modified with mPEG-SH, hyperbranched polyglycerol, or d-α-tocopheryl polyethylene glycol 1000 succinate.

17. A method for thermotherapy, comprising administering to a subject a composition comprising the fluorescent nanodiamond coated with porous polydopamine of claim 1 as an active ingredient.

18. Use of the fluorescent nanodiamond coated with porous polydopamine of claim 1 for thermotherapy.

19. Use of the fluorescent nanodiamond coated with porous polydopamine of claim 1 for drug delivery.

20. Use of the fluorescent nanodiamond coated with porous polydopamine of claim 1 for lesion labeling.

21. Use of the fluorescent nanodiamond coated with porous polydopamine of claim 1 for detecting nucleic acids.

22. A composition for preventing, treating, or diagnosing a disease, comprising the fluorescent nanodiamond coated with porous polydopamine of claim 1 as an active ingredient.

23. Use of the fluorescent nanodiamond coated with porous polydopamine of claim 1 for the prevention, treatment, or diagnosis of a disease.
